(19) United States
(12) Patent Application Publication
(10) Pub. No.: US 2006/0000360 A1
(43) Pub. Date: Jan. 5, 2006

(54) Indoor air circulation sterilizing device

(76) Inventors: Ming-Hwa Shou, Sindian City (TW); Ming-Song Chou, Sindian City (TW)

Correspondence Address: Yi-Wen Tseng, 4331 Stevens Battle Lane, Fairfax, VA, 22033, US

(21) Appl. No.: 10/879,114

(22) Filed: Jun. 30, 2004

(51) Int. Cl. A61L 9/20

(52) U.S. Cl. 96/224; 422/121

(57) ABSTRACT

An indoor air circulation sterilizing device is provided. The sterilizing device is installed to a return air inlet on the ceiling. The sterilizing device includes a frame body, at least three separator plates and a sterilizing lamp set. The frame body is composed of a first frame and a second frame. The first frame includes a plurality of containing grooves intermittently disposed thereon. The containing grooves can provide the separator plates to be disposed thereon. Several penetrating holes are formed on the separator plates. Each two adjacent separator plates are so disposed that the penetrating holes are mutually shifted. The sterilizing lamp set is disposed on the separator plates and is connected with the frame body. Therefore, there is provided a longer path for air to flow in the separator plates with a slower flowing speed, thereby sterilizing the bacteria in the air by employing the sterilizing lamp set.

### BACKGROUND OF THE INVENTION

The present invention relates generally to an indoor air circulation sterilizing device, and more particularly to a sterilizing device that is installed to a return air inlet on a ceiling. When the air passes through a separator plate, the flow path becomes longer and the flow speed becomes slower, thereby sterilizing the air by employing a sterilizing light set.

In order to rapidly cool down an indoor space with relatively more economic power consumption, most of the modern buildings are designed as air closed. Therefore, the airflow that passes through an air outlet of an air conditioner often contains a huge amount of malign bacteria. Once such bacteria are flown into an indoor space, it is often difficult to be exhausted within a short amount of time. People staying in such an environment are easily infected by the bacteria and bring such bacteria back to infect their family members, thereby widely spreading out such a contagious disease.

Conventional indoor air circulation device, as shown in FIG. 1, is disposed on a return air inlet on a ceiling. The conventional indoor air circulation device includes a frame 10a and a filter web 20a formed on the frame 10a. When an air conditioner blows cold air from an air outlet, the cold air is flown into a room. The cold air can absorb heat in the room and flow into the ceiling through the frame 10a and the filter web 20a, so as to circulate and cool the air in the room.

However, the conventional indoor air circulation device employs only the filter web 20a to filter out particles and the contagious bacteria. This can not completely eliminate the contagious bacteria in the air. On the other hand, when the air conditioner is off, the hot air adjacent the ceiling will flow down to the room. The malign bacteria hidden on the ceiling will be brought down to the room, thus severely damaging one's health.

Accordingly, the inventor of the present invention has devoted himself based on his many years of practical experiences to the development of a indoor air circulation sterilizing device that can resolve the problem discussed above.

### BRIEF SUMMARY OF THE INVENTION

The present invention is to provide an indoor air circulation sterilizing device. The sterilizing device employs a sterilizing lamp set and the formation of penetration holes on separator plates to provide a longer path for air to flow therethrough with slower flowing speed, thereby sufficiently sterilizing the air and killing the malign bacteria in the air.

In order to achieve the above and other objectives, the sterilizing device of the present invention includes a frame body, at least three separator plates and a sterilizing lamp set. The frame body is composed of a first frame and a second frame. The first frame includes a plurality of containing grooves intermittently disposed thereon. The containing grooves can provide the separator plates to be disposed thereon. Several penetrating holes are formed on the separator plates. Each two adjacent separator plates are so disposed that the penetrating holes are mutually shifted. The sterilizing lamp set is disposed on the separator plates and is connected with the frame body. Therefore, the objectives mentioned above can be achieved.

### BRIEF DESCRIPTION OF THE DRAWINGS

FIG. 1 illustrates a conventional indoor air circulation device.

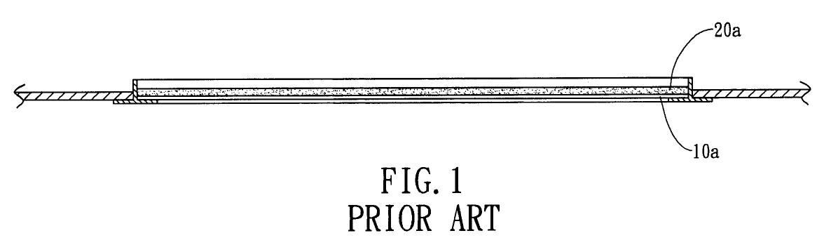

FIG. 2 is an explosive view of an indoor air circulation sterilizing device in accordance with one embodiment of the present invention.

FIG. 3 is a perspective view of the sterilizing device in accordance with one embodiment of the present invention.

FIG. 4 is a sectional view of the sterilizing device in accordance with one embodiment of the present invention.

FIG. 5 is a sectional view of the sterilizing device in accordance with one embodiment of the present invention, installed on a ceiling.

FIG. 6 schematically illustrates the combination of a first frame and a second frame, in accordance with one embodiment of the present invention.

FIG. 7 is a sectional view of a sterilizing device in accordance with another embodiment of the present invention.

### DETAILED DESCRIPTION OF THE INVENTION

In order to better understanding the features and technical contents of the present invention, the present invention is hereinafter described in detail by incorporating with the accompanied drawings. However, the accompanied drawings are only for the convenience of illustration and description, no limitation is intended thereto.

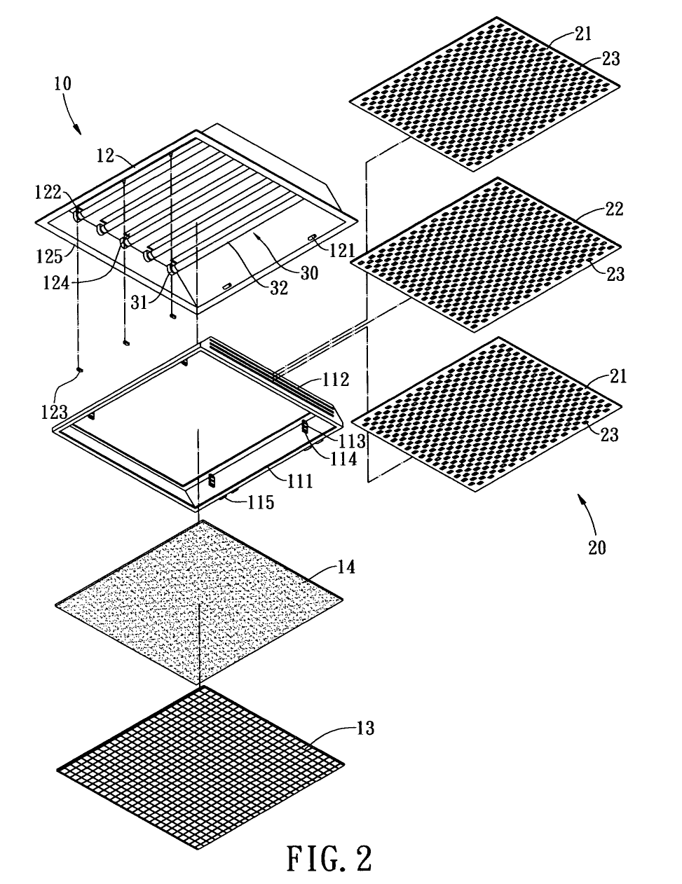

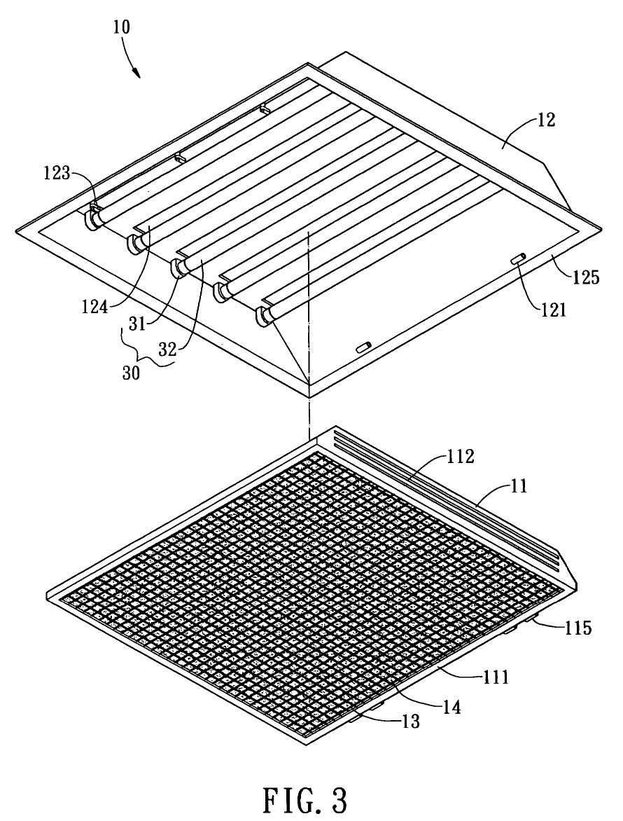

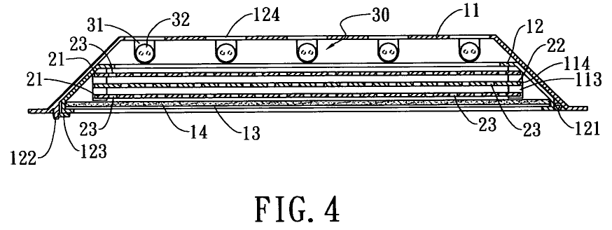

Referring to FIG. 2, FIG. 3 and FIG. 4, the explosive view, the perspective view and the sectional view of an indoor air circulation sterilizing device of the present invention are illustrated. The sterilizing device includes a frame body 10, at least three layers of separator plate 20, and a sterilizing lamp set 30.

The frame body 10 includes a first frame 11 and a second frame 12. The first frame 11 is composed of four side plates. Each side plate includes an inwardly extended extension plate 111 from the bottom rim of the side plate. The extension plate 111 provides a grating support member 13 to be disposed thereon. A filter web 14 is disposed on the support member 13. The filter web 14 can be an active carbon type or a high efficiency particulate air (HEPA) filter type. On the other hand, a plurality of containing grooves 112 is intermittently formed on the front plate of the first frame 11. A plurality of fastening plate 112 is protrusively formed on the inner side of the left and right side plates. A plurality of grooves 114 is formed on the fastening plate 113. The grooves are located corresponding to the containing grooves 112. A first ear 115 is formed on the outer side bottom rim of the right side plate. The second frame 12 is a hollow rectangular frame (not shown) or a trapezoid frame composed of four side plates. In this particular embodiment, the second frame 12 is a trapezoid frame. A second ear 121 is formed on the bottom rim of the right side plate. The second ear 121 and the first ear 115 are located corresponding to each other, each including a hole for penetrating a latch therethrough. A plurality of protrusive pillars 122 is formed on the periphery of the second ear 121. The protrusive pillar 122 includes a screw hole for fastening a knob 123 on the protrusive pillar 122 by means of a screw, for example. Furthermore, a plurality of tunneling paths 124 is formed on the top portion of the second frame 12. Four rectangular protrusive edges 125 are formed extending from the bottom rim of the second frame 12.

The separator plate 20 can be made of metallic materials. The separator plate 20 includes two first separator plates 21 and a second separator plate 22. Each of the separator plates 21, 22 includes a penetrating hole 23 formed thereon. The penetrating hole 23 can be in circular, rectangular or various other geometrical shapes. In this particular embodiment, the penetrating hole 23 is in rectangular shape. The first separator plate 21 is disposed in the containing groove 112 of the first frame 11 and the groove 114 of the fastening plate 113. The second separator plate 22 is sandwiched between the two first separator plates 21. The penetrating holes 23 of the first separator 21 and the second separator 21 are so disposed that they are mutually shifted.

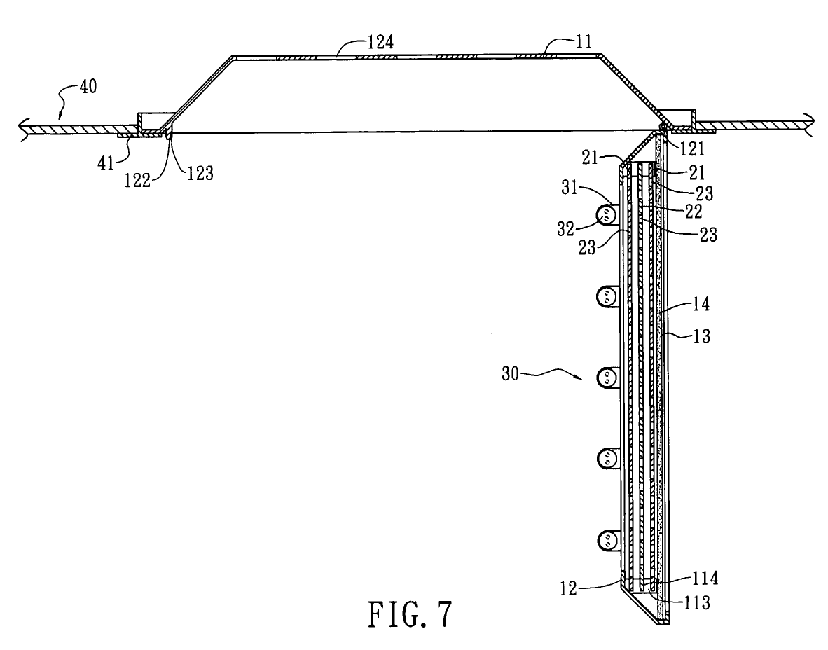

The sterilizing lamp set 30 includes a lamp holder and a lamp tube 32. The lamp holder 31 is disposed on the first frame 11, as shown in FIG. 7, or on two ends of the second frame 12. In this particular embodiment, the lamp holder 31 is disposed on the second frame 12. The lamp holder 31 provides a lamp tube 32 to be disposed thereon. The lamp tube in this particular embodiment is an ultraviolet sterilizing lamp, wherein the wavelength of the ultraviolet light emitted is preferably between 240 to 260 nm. In this particular embodiment, the UVC254 sterilizing lamp tube is employed.

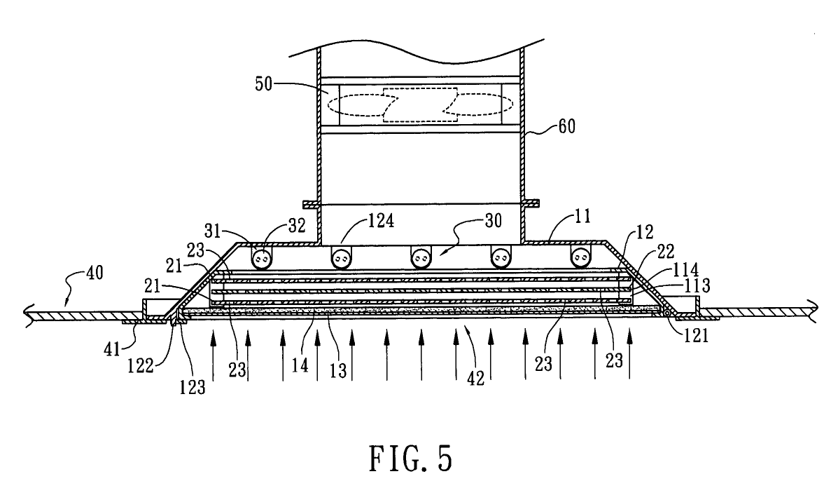

Referring to FIG. 5, a sectional view of the sterilizing device of the present invention is illustrated. The sterilizing device of the present invention is installed on the ceiling 40 of an indoor space, namely a room. The ceiling is composed of a plurality of vertically and horizontally arranged steel bars, thereby forming a rectangular return air inlet 42. The return air inlet 42 provides the protrusive edge 125 of the second frame 12 to be disposed thereon. When an air conditioner blows cold air toward an air outlet, the cold air starts absorbing heat in the room. After the heat absorption process, the air spontaneously flows up via convection and into the filter web 14 of the frame body 10. The air then enters into the penetrating holes 23 of the separator plates 20. Since the first separator plate 21 and the second separator plate 22 are so disposed that they are mutually shifted, there is provided a longer path for the air to flow in the separator plates with a slower flowing speed. The bacteria in the air can thus be exposed to the ultraviolet light from the sterilizing lamp set 30 with sufficient amount of time. In addition, since the penetrating holes 23 are so disposed that they are mutually shifted, the ultraviolet light emitted from the sterilizing lamp set 30 can thus be blocked from entering the room, thereby preventing people from being hurt. Moreover, a fan 50 and/or a return air tube 60 can be disposed on the tunneling path 124 of the second frame so as to enhance the suction of air for achieving better sterilizing effect.

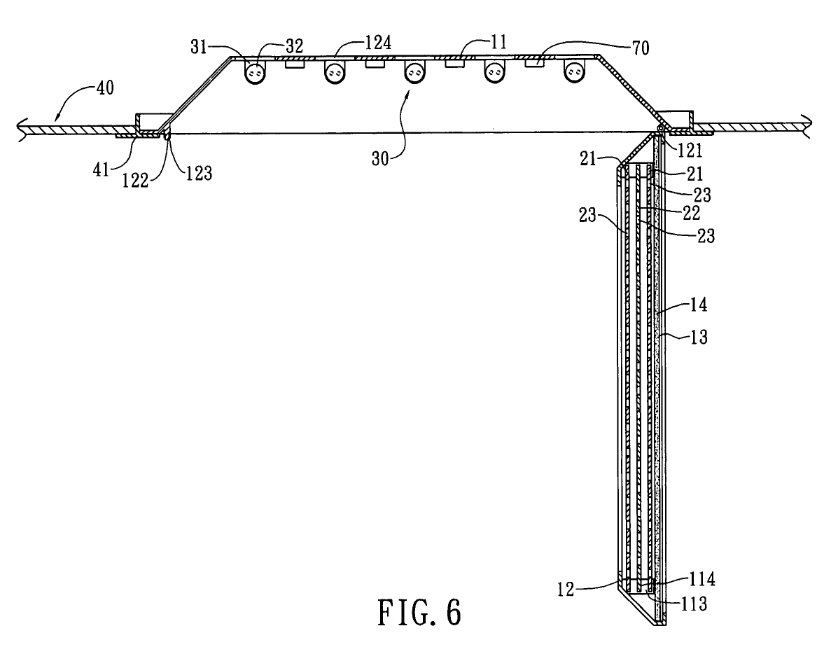

Referring to FIG. 6, a combination of the first frame and the second frame is schematically illustrated. Once the intensity of the ultraviolet light emitted from the ultraviolet lamp tube 31 is below 40 uw/cm*cm, the sterilizing effect can never be achieved regardless of how much time the air is exposed. Therefore, a lamp tube monitoring device 70 can be disposed on one side of the second frame 12 so as to detect the intensity of the ultraviolet light. The monitoring device 70 is electrically coupled to the circuitry of the sterilizing lamp set 30. Once the intensity of the ultraviolet light is below the value given above, it indicates that the lamp tube 31 needs to be replaced. Therefore, when such occasion occurs, the monitoring device 70 emits a lamp replacement signal. One can turn the knob 123 at the bottom rim of the second frame 12 about the first ear 115 (referring to FIG. 2) of the first frame 11, so as to perform the replacement of the lamp tube 31.

In summary, the sterilizing device of the present invention indeed contains patentable subject matter that is consistent with the utility, novelty and non-obviousness requirements as set forth in the patent law. Therefore, a grant of letters patent for the present invention is respectively requested.

Since, any person having ordinary skill in the art may readily find various equivalent alterations or modifications in light of the features as disclosed above, it is appreciated that the scope of the present invention is defined in the following claims. Therefore, all such equivalent alterations or modifications without departing from the subject matter as set forth in the following claims is considered within the spirit and scope of the present invention.

What is claimed is:

1. A sterilizing device, comprising:
a frame body including a first frame with a plurality of containing grooves intermittently disposed thereon and a second frame;
a plurality of separator plates respectively disposed in the containing grooves, each separator plate comprising a plurality of holes formed thereon, wherein the holes on each two adjacent separator plates are mutually shifted; and
a sterilizing lamp set disposed on the frame body and above the separator plates,
whereby a longer path is provided for air to flow in the separator plates with a slower flowing speed so as to sterilize bacteria in the air by the sterilizing lamp set.

2. The sterilizing device as recited in claim 1, wherein the first frame and the second frame are pivotally connected.

3. The sterilizing device as recited in claim 1, further comprising an extension plate formed on a bottom rim of the first frame to provide a supporting member connected thereon.

4. The sterilizing device as recited in claim 3, further comprising a filter web fastened on the supporting member to be an active carbon filter web and a high efficiency particulate air filter web.

5. The sterilizing device as recited in claim 1, further comprising a fan connected to the top surface of the second frame.

6. The sterilizing device as recited in claim 1, further comprising a return air tube connected to the top surface of the second frame.

7. The sterilizing device as recited in claim 1, further comprising a return air tube and a fan connected to the top surface of the second frame.

8. The sterilizing device as recited in claim 1, further comprising a lamp tube monitoring device disposed on one side of the second frame to electrically couple a circuitry of the sterilizing lamp set.

9. The sterilizing device as recited in claim 1, wherein the separator plates are made of metallic material.

10. The sterilizing device as recited in claim 1, wherein the holes on the separator plate have circular or rectangular shapes.

11. The sterilizing device as recited in claim 1, wherein the sterilizing lamp set is connected to the first frame or the second frame.

12. The sterilizing device as recited in claim 1, wherein the sterilizing lamp set comprises a lamp holder and a lamp tube.

13. The sterilizing device as recited in claim 12, wherein, the lamp tube is an ultraviolet sterilizing lamp.
